(19) United States
(12) Patent Application Publication
(10) Pub. No.: US 2005/0281872 A1
(43) Pub. Date: Dec. 22, 2005

(54) Enhanced compliance antiviral medicaments and methods of manufacture and use

(76) Inventors: Peter Summerville, Altamonte Springs, FL (US); Martin A. Nassif, Longwood, FL (US)

Correspondence Address: PRESTON GATES & ELLIS LLP, 1900 MAIN STREET, SUITE 600, IRVINE, CA, 92614-7319, US

(21) Appl. No.: 10/870,885

(22) Filed: Jun. 16, 2004

(51) Int. Cl. A61K 31/7056; A61K 9/48; A61K 9/20

(52) U.S. Cl. 424/451; 514/43; 424/464

(57) ABSTRACT

Reduced gastrointestinal side effect medicaments for treating viral infection in a patient suffering therefrom are provided comprising at least 500 mg of antiviral compound in an oral dosage form that can be administered in effective total daily dosages of antiviral compound ranging from 1000 mg to 2000 mg. The reduced gastrointestinal side effect medicaments enhance patient compliance with long term, multi-dose treatment regimens by reducing physiological and psychological side effects that can cause reductions or discontinuations of antiviral therapy. Exemplary antiviral compounds include nucleoside analogues such as ribavirin, levovirin, and viramindine which are effective when combined with interferon to treat acute or chronic viral infections including hepatitis, and particularly hepatitis C. Associated methods for the production and use of the medicaments also are provided.

### FIELD OF THE INVENTION

The present invention generally relates to the field of antiviral medicines and to treatments for viral infection in humans. More particularly, the present invention relates to antiviral medicaments such as ribavirin formulated to reduce gastric distress in patients undergoing antiviral therapy and to associated methods of production and use.

### BACKGROUND OF THE INVENTION

It has long been a goal of modern medicine to provide effective treatments for viral infections and human disease conditions associated with or caused by viral infections. Because of the fundamental genetic nature of viruses contemporary antibiotics are completely ineffective at relieving or resolving viral infections. In most circumstances viral infections are simply treated with palliative measures to reduce the impact of outward symptoms such as pain and fever in an effort to improve the quality and character of the infected patient's life. The resolution of the viral infection itself is left to the patient's natural immune response. Antibiotics may be given to avoid the possible onset of secondary bacterial infections which can complicate treatment. In many cases a patient's immune system will recognize and resolve a common viral infection in the matter of a few weeks. In other cases, the impact of a viral infection is more permanent and severe. The viral infection may become chronic and never be completely resolved. As a result, the infected patient's life is forever changed. In the worst cases, the initial viral infection will become chronic and active, leading to ongoing and progressively worsening disease conditions which may ultimately lead to organ failure and death.

Viral hepatitis is an example or such a worse case scenario. Viral hepatitis is any form of liver inflammation that is caused by a viral infection. It is a very difficult disease to resolve as the natural human immune response is ineffective at controlling or eliminating the underlying viral cause. There are five major types of viral hepatitis known today. These have been identified as hepatitis A, B, C (non-A, non-B), D, and E as characterized by the different viral causative agents involved. An additional virus causing hepatitis recently has been identified as hepatitis G and a seventh virus, hepatitis F is now suspected, but not confirmed as causing hepatitis. Each of these viruses is identified and distinguished from one another by its respective surface and core antigens and by the associated antibodies developed and found in the circulating blood of an infected individual utilizing molecular biology techniques.

The presence of these known distinguishing antigens and antibodies can be tested for using a variety of proven and reliable antigen-antibody blood tests. Depending upon the causative viral agent involved in an individual patient, the transmission, course, and severity of hepatitis can vary significantly. This is true of virtually all viral diseases. Different viral subtypes of the same viral species can produce differing courses of disease and can respond differently to treatment or therapy. Thus, for purposes of explanation and not for purposes of limitation, the subject invention will be discussed in the exemplary context of viral hepatitis as this context is illustrative of the principles of the present invention.

For example, hepatitis A is caused by the hepatitis A virus (HAV) and is commonly called “infectious hepatitis”. Relatively speaking, it is usually a mild disease. HAV is most often spread by contaminated food and water, but may be spread through sexual contact. Hepatitis B is commonly called “serum hepatitis” and is caused by the hepatitis B virus (HBV). Serum hepatitis can be mild or severe and it can be acute (of limited duration) or chronic (ongoing). It is usually spread through sexual contact with an infected person, through contact with infected blood, through intravenous drug use, or from mother to child at birth.

Hepatitis C used to be known as “non-A, non-B hepatitis” reflecting the development of the understanding and diagnosis of hepatitis over the years. It has been widely present in the United States population for decades and is caused by what is now known as the hepatitis C virus (HCV). Hepatitis C is spread mainly through contact with infected blood and thus poses a significant problem to healthcare workers and to recipients of blood products and organ donations. Transmission through sexual contact as well as through sharing tattoo or drug needles also is a possibility. However, at present the origin of nearly half of HCV infections cannot be traced.

Hepatitis C is generally a mild disease in its early, acute stage, but it is more likely to lead to chronic liver disease than hepatitis B. This is a very serious problem as chronic liver disease can lead to possible liver failure and to the eventual need for a liver transplant. In fact, it is believed that chronic liver disease states develop in more than 80% of patients with HCV infection and as many as 20% of patients with chronic hepatitis C will develop liver failure or liver cancer. Because the disease process is virtually undetectable clinically without any signs or symptoms of the disease until the liver is severely damaged, it is very difficult to determine a true number of those individuals that may be infected with HCV. Current estimates indicate that there may be as many as five million people in the United States with hepatitis C that do not know they are infected.

Hepatitis D is known as “delta hepatitis” and is caused by the hepatitis D virus (HDV). This disease only occurs in those individuals with HBV in their blood from a past or simultaneously occurring infection. Most cases occur among those who are frequently exposed to blood and blood products and many cases occur among drug users who share contaminated needles. Some experts believe that HDV transmission may occur through sexual contact. Further research is needed to confirm this understanding.

Hepatitis E is quite rare in the United States and is caused by the hepatitis E virus (HEV). Currently, HEV is understood as being another type of non-A, non-B hepatitis. The role of person to person transmission of this virus is unclear, but it is believed to be spread most often through fecally contaminated water. At present, there are no antigen or antibody tests widely available to accurately detect HEV. Similarly, the recent identification and understanding of hepatitis G and the suspected hepatitis F have not developed to the point where a lot is known about either of these viruses.

With this understanding of viral hepatitis it should be appreciated that HCV infection and the associated hepatitis C complications are becoming a very serious problem for contemporary society. Some estimates indicate that hepatitis C induced liver damage may ultimately be responsible for more annual deaths in the United States than are associated with HIV infection and AIDS. No vaccine has yet been developed to prevent hepatitis C in persons exposed to the virus. At the outset of HCV infection only about 25% of patients exhibit symptoms of acute (rapid onset) hepatitis. These symptoms include fatigue, muscular aches, loss of appetite and low-grade fever. The remaining 75% experience minimal or no symptoms. As the hepatitis becomes chronic, most infected individuals remain without symptoms, despite progressive liver inflammation, necrosis (liver cell death) and fibrosis (scarring). As the disease progresses over time with more advanced scarring of the liver, or cirrhosis, HCV patients experience muscle wasting and generalized weakness along with easy bruising. Later HCV symptoms associated with increasing cirrhosis include fluid retention, swelling of the lower extremities, fluid in the abdominal cavity, dilated esophageal veins and internal bleeding, mental confusion, sleepiness and liver cancer, which itself can cause abdominal pain, weight loss and fever.

Because of the difficulty of treating viral hepatitis considerable public health efforts have been devoted to the development of effective diagnostic tests for viral hepatitis and to educating the public about the modes of viral transmission in order to prevent or at least to reduce viral transmission and infection rates. Patients are advised to rest, eat a balanced diet with little fat, and to limit or avoid alcohol or any medication that can cause liver damage. Though effective, knowledge and education cannot eliminate that fact that many viral carriers, particularly those with HCV, have no idea they have the disease and therefore do not feel at risk or at need to change risky behaviors. Thus, not only do they run the risk of developing serious complications such as liver cancer, they also run the risk of passing the virus on to others.

Recently, effective antiviral therapies originally developed in conjunction with the global response to the AIDS pandemic have been applied to the treatment of viral hepatitis with notable success. The ultimate goal of any antiviral therapy is to eliminate the viral infection. In addition to eliminating the presence of virus, with hepatitis the goals of antiviral therapy include improving or normalizing liver tests, preventing disease progression to cirrhosis and liver cancer, prolonging patient survival, and improving the quality of the infected patient's life and the lives of those associated with them.

Such a modern antiviral therapy is the use of interferon. Interferon is a natural body protein now produced in large amounts through genetic engineering and has been demonstrated to improve the outlook for many AIDS patients and for HCV patients with chronic hepatitis C infection. In HCV it has been shown to reduce or lessen patients' symptoms and to improve liver function. A newer form of interferon known as pegylated interferon is also being used. Pegylated interferon functions in much the same way as regular or normal interferon, but reduces the frequency of drug administration necessary for effective treatment from daily injections to weekly injections. As further evidence of the difficulty of treating chronic viral diseases, not all patients respond to interferon therapies and other patients get less benefit the longer they take interferon. Still, nearly half of the patients using a high dose of interferon for six months have responded positively.

In the case of treating HCV infection with antiviral compounds such as interferon, an optimal or “sustained” response to antiviral therapy is defined as the absence of detectable HCV RNA six months after treatment is stopped. Many of the individuals demonstrating this optimal response will remain in remission indefinitely after treatment is completed with no detectable HCV RNA in their blood or liver. In these cases follow-up liver biopsies show marked reductions in liver inflammation and scarring. In contrast, half of the patients who do respond well initially to HCV antiviral therapy will relapse after therapy is stopped. The HCV virus again becomes detectable within six months after discontinuing therapy. Further, a significant number of patients are “non-responders” and have detectable HCV RNA even during therapy. In spite of this, the value of a completed antiviral therapy regimen is demonstrated by the fact that even relapsers and non-responders may show an improvement in liver injury and scarring after antiviral therapy.

A recent improvement in antiviral therapy is to use another new antiviral medication in conjunction with conventional interferon or pegylated interferon to treat viral infections such as hepatitis, particularly hepatitis C. This new antiviral agent is called ribavirin. It is a “nucleoside analogue” which is a man made molecule that mimics the biochemical units that make up natural genetic material (DNA and RNA). The therapeutic goal of the use of nucleoside analogues is to trick the infecting virus to use the nucleoside analogues as one or more of the building blocks of genetic material instead of the normal genetic building blocks the virus would use to create copies of itself. This results in slowing down viral replication because the virus ends up making useless genetic material that cannot infect additional cells in the infected patient.

Ribavirin alone is not effective in treating hepatitis C. However, when combined with interferon, if tolerated by the patient, ribavirin does increase positive response rates to treatment. The combination of ribavirin and interferon is associated with considerably more side effects than interferon treatment alone and this has contributed to a significant decline in patient compliance and the resultant effectiveness of treatment. Recent reports indicate that up to 20% of patients receiving ribavirin required a reduction in dose or even a discontinuation of therapy because of side effects. The most common reasons for discontinuing therapy were related to psychiatric, systemic (e.g. fatigue, headache), or gastrointestinal adverse reactions.

In effect, the patients felt worse during therapy than they did before therapy. As a result, they were deterred from continuing treatment and from possibly resolving or at least improving their disease conditions. More importantly, when therapy is held or stopped because the side effects are too severe, the virus remaining in the infected patient may develop resistance to the therapy, rendering subsequent treatment ineffective. Thus, patient non-compliance and the discontinuation of antiviral therapy due to side effects remains a significant problem to the field of antiviral therapy.

Though it is possible to reduce the flu-like symptoms of headache and fatigue associated with antiviral therapy utilizing secondary therapies such as analgesics, contributing to the significant issue of patient non-compliance is the gastrointestinal discomfort associated with ribavirin. Multiple doses of ribavirin are required on a daily basis as part of the normal combined ribavirin interferon antiviral treatment regimen. Because the global pharmaceutical market offers ribavirin in only a 200 mg dose product, the infected patient may be required to swallow 6 to 8 of these 200 mg doses daily in a rigorously scheduled multiple dose protocol. Regardless of the type of ribavirin capsules or tablets swallowed with each of the multiple daily doses required, the lining of the patient's stomach will bleed to varying degrees each and every time the capsules or tablets are swallowed. This results in considerable discomfort and nausea making it a challenge to keep the patients on the twelve to eighteen month average course of therapy necessary to achieve the optimum antiviral response. To date, reducing or discontinuing therapy is the only effective way to reduce these gastrointestinal side effects.

Accordingly, in spite of the success of recent antiviral therapy regimens in treating virally induced diseases like HCV and HIV, a significant need remains to reduce the side effects associated with ribavirin therapy in general, in order to increase patient compliance with the necessary treatment protocols and to improve the likelihood of successful treatment and resultant disease eradication. It is an object of the present invention to address this need.

### SUMMARY OF THE INVENTION

In a broad aspect the present invention provides reduced gastrointestinal side effect medicaments for treating viral infections as well as associated methods for the production and use of these novel medicaments. By reducing gastrointestinal bleeding and the resultant patient discomfort from having to repeatedly take multiple dosages of stomach irritating medications every day, the medicaments of the present invention make it easier for patients needing antiviral therapy to comply with the rigorous treatment protocols necessary to effectively combat serious acute or chronic viral infections. The enhanced patient compliance provided by the reduced side effect medicaments and methods of the present invention is further augmented by the practical ease and resultant patient psychological benefit provided by the direct teachings of the present invention which reduce the number of doses per day that the patient must take to achieve an effective total daily dose. Because the average course of antiviral therapy requires at least six months and often up to twelve and even eighteen months of strictly controlled daily medication dosing, the cumulative benefit to patient compliance provided by the present invention's medicaments and methods which require fewer doses of lower gastrointestinal side effect, yet equally effective antiviral medications cannot be overestimated.

Accordingly, it is a primary object of the present invention to provide patients needing antiviral therapy with novel medicaments and methods which reduce the gastrointestinal side effects known to be responsible for reductions in dosage or even complete discontinuations of antiviral therapy. These and additional objects are achieved by the medicaments and methods of the present invention which provide for the necessary 1000 mg to 2000 mg daily dosages of antiviral compounds currently being prescribed. These dosages are determined based upon the infected patient's weight, viral load, blood count, and side effects as known in the art. As a result, the present invention makes it possible to effectively combat chronic viral infection while significantly reducing the gastrointestinal side effects and the number of individual doses the patient must take each day in order to comply with the treatment regimen.

Thus, in accordance with the teachings of the present invention, reduced gastrointestinal side effect medications for treating viral infections in patients suffering therefrom are provided which comprise an oral dosage form medicament of at least 500 mg of antiviral compound. Exemplary antiviral compounds within the scope and teachings of the present invention include nucleoside analogues such as ribavirin, levovirin (a mirror image of the ribavirin molecule), and viramindine. Additional nucleoside analogues and antiviral compounds, either alone or in combination, are also within the scope of the present invention. When taken in combination with interferon, ribavirin has been proven to be effective in the treatment of viral hepatitis, particularly hepatitis C, and HIV. Similarly, medicaments having at least 600 mg of antiviral compound are also within the scope and teachings of the present invention.

In contrast to the teachings of the art which provide only 200 mg capsules and 40 mg liquid doses of antiviral compounds, the present invention significantly reduces the number of doses that must be swallowed by the infected patient. The prior art antiviral compounds require the infected patient to swallow six to eight of these 200 mg doses daily in divided doses to achieve the 1000 mg to even as high as 1600 mg to 2000 mg total daily dosages of antiviral compound necessary to combat the viral infection. The present invention significantly reduces the number of doses that must be swallowed by the infected patient to less than four per day, and in many cases to only two or three individual dosages per day in a single administration. Thus, the present invention significantly reduces the number of stomach irritating doses that must be swallowed by the individual undergoing antiviral therapy.

This has the direct positive benefit of reducing the bleeding in the patient's stomach lining which invariably results from swallowing multiple individual drug capsules or tablets which physically and mechanically irritate the patient's stomach lining causing bleeding. The present invention also eliminates the need to divide the total daily dosage of antiviral compound into multiple, multi-capsule doses spread through out the day. By simplifying the dosing regimen and by reducing the gastrointestinal side effects associated with stomach bleeding from swallowing pills, the present invention enhances patient compliance and improves the likelihood of a complete and successful multi-month antiviral treatment regimen.

In accordance with the teachings of the present invention the medicaments can be an oral dosage capsule or tablet and may include one or more pharmaceutically acceptable excipients or carriers. The reduced gastrointestinal side effect medicaments of the present invention can be produced as capsules or as tablets utilizing any appropriate pharmaceutical manufacturing technique as known in the art. For example, those skilled in the art will appreciate that a wide variety of automated tablet forming machines are commercially available and readily adaptable to the teachings of the present invention. Similarly, manual and automated capsule loading machines are widely available and can be adapted to produce the medicaments of the present invention utilizing appropriately sized gelatin capsules available through conventional sources known in the art of drug manufacturing. As an added benefit, the antiviral compounds utilized to practice the present invention do not have to be chemically modified to function within the scope and teachings of the present invention.

For example, one embodiment of the present invention particularly well suited for use in connection with the treatment of viral hepatitis, particularly hepatitis C, is a medicament formed of 500 mg of an antiviral compound such as ribavirin in an oral dosage form. This oral dosage form can be a capsule or a tablet and may include one or more pharmaceutically acceptable excipients or carriers such as microcrystalline cellulose, lactose monohydrate, croscarmellose sodium, and magnesium stearate. When formed as a capsule, the capsule may be gelatin or other pharmaceutically acceptable materials as known in the art.

An alternative embodiment of the present invention that is well suited for use in treating viral hepatitis, particularly hepatitis C, is a medicament formed of 600 mg of a nucleoside analogue such as ribavirin in an oral dosage form such as a capsule or tablet. This alternative embodiment may also include a pharmaceutically acceptable excipient.

Further, by utilizing the teachings of the present invention a method of enhancing a virus infected patient's compliance with a long term, multi-dose antiviral therapy regimen also is provided. This method includes, in a broad aspect, the steps of providing a reduced gastrointestinal side effect medicament having at least 500 mg of antiviral compound in an oral dosage form and then administering an effective total daily dosage of this oral dosage form to the infected patient for a predetermined period of time consistent with normal antiviral treatment utilizing the antiviral compound. The antiviral compound can be a nucleoside analogue such as ribavirin and the effective total daily dosage ranges between 1000 mg per day to 1600 mg per day or even to 2000 mg per day. Further in accordance with the teachings of the present invention, the predetermined period of time will range from six to eighteen months.

Alternatively, the method of enhancing a virus infected patient's compliance with a long term, multi-dose antiviral therapy of the present invention also can include the additional step of administering an effective daily or weekly dosage of interferon in combination with the effective total daily dosage of antiviral compound. As those skilled in the art will appreciate, the interferon can be conventional or pegylated and the interferon dosage and administration will be adjusted appropriately, as known in the art.

Additionally, the present invention also provides a method for manufacturing the reduced gastrointestinal side effect medicaments of the present invention for the treatment of viral infection in an individual suffering therefrom. The method comprises the step of packing at least 500 mg of an antiviral compound in an oral dosage form. The oral dosage form can be a capsule or a tablet. The antiviral compound can be an nucleoside analogue such as ribavirin.

Further novel features and other objects of the present invention will become apparent to those skilled in the art from the following Detailed Description and the appended claims.

### DETAILED DESCRIPTION OF THE INVENTION

Although specific embodiments of the present invention will now be described in detail, it should be understood by those skilled in the art that such embodiments are by way of example only and not of limitation. They are merely illustrative of but a small number of the many possible embodiments of the present invention. Various changes, modifications, and adaptations are available to one skilled in the art to which the present invention pertains and are within the scope of the present invention as will be discussed in detail below and as further defined in the appended claims.

While the human immune systems does an amazing and complex job of identifying and resolving bacterial and viral infections on a daily basis, many viral infections are virtually impossible to eliminate. Within the past few decades, as the science of molecular biology and medicine have merged in response to the AIDS pandemic, a number of very promising antiviral therapies have emerged. The diagnosis of HIV infection is no longer a death sentence because effective antiviral treatments have and are continuing to be developed that are capable of slowing the viral replication cycle and the progression of the disease. These same antiviral compounds have also been utilized with recent success against other chronic and debilitating viral infections such as those responsible for hepatitis.

Unfortunately, these antiviral therapies require the infected patient to follow very strict drug dosing protocols. Large quantities of antiviral drugs and drug combinations must be taken every day at specific times for months on end without interruption. Eating patterns must be adjusted to avoid conflict with the ingestion and subsequent bioavailability of the antiviral medicines or because specific medicines must be taken in combination with food. Side effects are common. Entire lifestyles must be changed to adapt to these dosing regimens and to the often debilitating side effects produced by the antiviral compounds.

In many individuals undergoing antiviral therapy these changes and side effects are simply too much. The therapies must be adjusted to reduce the side effects to tolerable levels and in many cases the therapy must be stopped altogether. It is a primary object of the present invention to reduce at least some of the side effects associated with antiviral therapy in order to enhance or improve patient compliance with the rigorous antiviral treatment dosing protocols. Doing so will enable the antiviral treatments to be tolerated by the patients for longer periods of time at the appropriate drug dosage levels in order to give the therapies the greatest chances of success at resolving the patients' infections.

To assist in the understanding of the present invention, antiviral therapy will be described in the broad context of the treatment of viral hepatitis, particularly the treatment of hepatitis C caused by the hepatitis C virus (HCV). It will be appreciated by those skilled in the art that the present invention is not limited to the treatment of hepatitis C or to the treatment of viral hepatitis alone. Rather, the present invention is directly applicable to any antiviral therapy where gastrointestinal side effects have the potential of limiting patient compliance with treatment protocols. The treatment of hepatitis C is a useful vehicle to illustrate the teachings of the present invention as there is significant experience in the art with the antiviral treatment of hepatitis C. Contrasting this prior art experience with that of the present invention demonstrates the features and benefits of the present invention over the prior art.

It is impossible to kill a virus with a drug. Disease causing bacterial pathogens are living cells that are susceptible to antibacterial compounds, such as penicillin, that can poison or interrupt the bacterial metabolisms. In contrast, viruses are not living cells. They are actually complex molecular structures of proteins and genetic material that, once inside a patient's cell, convert the infected cell's metabolism away from its normal life sustaining activities into viral production factories, eventually killing the infected cell. Viruses have no metabolic pathways of their own that can be interrupted with conventional antibacterial drugs. As a result, it is extremely difficult to interrupt viral replication without interrupting the patient's own cellular metabolism. Until recently, these practical limitations prevented contemporary medicine from doing more than treating the symptoms of viral infection in an effort to improve patient comfort and the quality of life while the patient's own immune systems did battle with the infecting virus.

Recently, medical science has been able to develop compounds known as “nucleoside analogues” which mimic the natural building blocks of genetic material that the infected patient's cells utilize to construct new viral particles. As presently understood, instead of using naturally occurring nucleosides to create new viruses, virally infected cells treated with nucleoside analogues build non-functional viral genetic material utilizing the nucleoside mimicking analogues in place of these natural genetic building blocks. The resultant non-functional genetic material stops the virus from reproducing and infecting additional cells in the infected patient's body or from being transmitted to new, uninfected individuals by the infected patient. Though not every virus or viral subtype currently is susceptible to antiviral therapy, in many cases complete remission of specific viral diseases can be obtained.

Ribavirin is an exemplary nucleoside analogue that is illustrative of the teachings of the present invention. Ribavirin is available from the Schering-Plough corporation under the brand name Rebetol® and recently became available from the Hoffman-La Roche corporation under their brand name Copegus®. Generic drug manufacturers also are producing ribavirin from bulk drug manufactured in China. Additional exemplary nucleoside analogues within the scope and teachings of the present invention are levovirin, a mirror image of the ribavirin molecule, and viramindine, a ribavirin prodrug that is preferentially absorbed by liver cells. Vertex Pharmaceuticals is also developing its own similar antiviral drug currently known as VX497. Though competing on price in a global marketplace, all manufacturers currently producing ribavirin only make the drug available in a 200 mg dosage form, typically as an oral capsule. Oral solutions of ribavirin are available for children, but they contain an even lower dosages on the order of only 40 mg. As known in the art, these dosage limits are believed to be necessary to allow dosing flexibility based on patient body weight, blood counts, viral load, and the side effects experienced. They are believed to make the compounds easier to tolerate and absorb in effective total daily dosages that are significantly above 200 mg.

For example, patients suffering from viral hepatitis, particularly hepatitis C, are potential candidates for antiviral therapy. Balancing the costs, effectiveness, and side effects of current antiviral therapy, the National Institute of Health recommends antiviral treatment for infected individuals who are at the greatest risk of developing cirrhosis of the liver. These patients exhibit all of the following symptoms: persistent ALT elevation (a liver enzyme circulating in the blood evidencing liver inflammation); detectable HCV RNA (the hepatitis C viral gene circulating in the patient's blood); evidence of fibrosis (scarring) of the liver upon biopsy; and, evidence of at least moderate inflammation and liver cell injury (necrosis) on liver biopsy. Additionally, because about 85% of patients with acute hepatitis C will progress to chronic liver disease it is recommended that these individuals be treated with antiviral therapy to prevent the infection from becoming chronic.

Once the patient has been identified as an antiviral treatment candidate the next step is to determine an appropriate dosage and treatment protocol for the individual patient. Early antiviral treatment for HCV relied upon the use of interferon alpha alone given as subcutaneous injections three times per week. More recently, pegylated interferon (interferon modified to remain in the body longer) has been used on a once weekly injection schedule. Though successful in resolving infection in some patients, even better results were obtained with a combination of interferon and ribavirin. The recommended patient dosage of ribavirin is 15 mg/kg of body weight per day orally, divided into two doses per day. For example, patients under 75 kilograms (about 165 pounds) typically are prescribed two 200 mg ribavirin capsules in the morning and three 200 mg capsules at night for a total daily dosage of 1000 mg. Patients over 75 kilograms typically are prescribed three 200 mg capsules in both the morning and the evening for a total daily dosage of 1200 mg. These recommended total daily dosages can be increased as determined necessary by the treating physician and can range as high as 1600 mg to 2000 mg per day. Ribavirin can be administered without food, but should be administered in a consistent manner with respect to food intake. The recommended duration of this combination interferon ribavirin treatment for previously untreated patients is six to twelve months and is individualized to the patient depending on baseline disease characteristics, response to therapy, and tolerability of the regimen.

After six months (24 weeks) of treatment the patient's virologic response is assessed utilizing well known antigen/antibody techniques. The optimal response to antiviral treatment is called a “sustained response” and is defined as the absence of detectable HCV RNA in the patient's blood after six months of treatment, the completion of an entire course of antiviral therapy. The testing protocols for identifying HCV RNA and other markers of HCV infection are well known in the art and are incorporated herein by reference. Most of these sustained response patients will remain in remission with no signs of the disease for the foreseeable future, or at least for an indefinite period of time. In addition to the absence of detectable HCV RNA in the blood or liver, follow-up biopsies on patients in remission show a marked reduction in inflammation and a regression of fibrosis or scarring in the liver, further confirming the efficacy and the positive effects of antiviral treatment.

Alternatively, in a number of cases the HCV RNA becomes undetectable during therapy, but becomes detectable again within three to six months following the end of therapy. This type of response is called a “relapse”. Finally, there are “non-responders”. These are patients who have detectable HCV RNA even during antiviral therapy. Relapsers and non-responders may be retreated with the same or longer antiviral protocols, but success rates vary. An improvement in liver injury and scarring can be seen in some cases, but it is not as substantial as that observed in sustained responders.

It should be noted that HCV response to antiviral therapy varies with the genotype of the hepatitis C virus involved. HCV genotypes are hepatitis C viruses with slight genetic variations that make them distinguishable from one another. Patients with genotype 2 or 3 are more likely to respond to antiviral therapy than are those with genotype 1. For example, for combined ribavirin and interferon therapy the overall sustained response rate after 12 months of therapy is about 40%. However, for genotype 2 or 3 patients the sustained response rate is closer to 60%, while it is only about 30% for patients infected with genotype 1.

When and if the HCV infection is eradicated, as evidenced by the absence of detectable HCV RNA in the patient's blood, the treated patient can expect a favorable outcome. Elimination of the viral infection normally improves liver tests (high blood levels of liver enzymes known as ALT and AST are indicative of liver inflammation) and the histology (microscopic appearance) of the liver. Additionally, elimination of HCV infection may prevent the progression of cirrhosis and liver cancer and will improve the quality of the patient's life as well as prolong the patient's survival. These are the ultimate goals of antiviral therapy and are consistent with the goals of the present invention which reduces the gastrointestinal and associated psychological side effects of antiviral therapy without modifying the antiviral compounds themselves and without lowering or raising the total daily dosage.

Though clearly offering the promise of potentially improved health and longevity to patients suffering from acute and chronic viral infections, antiviral therapy is not without side effects. These side effects can range from psychological issues such as depression to physical issues such as fatigue, muscle aches, and other flu-like symptoms, as well as to gastrointestinal issues including nausea, loss of appetite, indigestion, and vomiting. These side effects can be so debilitating as to result in the infected patients being unable to or refusing to follow the treatment dosing and duration protocols. This is where the present invention shows its significant and unexpected benefits of enhancing patient compliance through the reduction of gastrointestinal side effects as well as by simplifying the daily dosage regimen and reducing the psychological side effects of multi-dose, long duration therapy.

Patient compliance can never be assumed. Many factors contribute to patient noncompliance. It is generally believed that the greater the number of drugs a patient is taking the higher the risk of noncompliance. The same is true for the frequency of drug administration. The more often a drug must be taken the more likely the patient will not want to be inconvenienced by taking it. Current studies indicate that up to 20% of patients receiving ribavirin antiviral therapy have required a reduction in dose or the complete discontinuation of therapy because of side effects. Many of these side effects can be handled with secondary therapies such as analgesics and pain relievers to reduce discomfort and with psychological counseling or group therapy to reduce depression and other psychological disorders. However, no one in the art has been able to resolve the gastrointestinal side effects of antiviral therapy. Early efforts at doing so involved the use of antacids. However, this practice is discouraged because taking antacids containing magnesium, aluminum, and simethicone resulted in a 14% decrease in available ribavirin.

In contrast to the prior art, the present invention significantly reduces gastrointestinal side effects by reducing the quantity of oral dosages that must be swallowed throughout the day to achieve the effective total daily dosage prescribed. Moreover, the present invention also provides the additional psychological benefit of reducing both the number of capsules, tablets, or pills that must be taken at each scheduled drug administration as well as reducing the frequency of drug administrations per day necessary to achieve the total daily dosage. These advantages and benefits are achieved by the present invention without decreasing the amount of drug available in the patient's circulating blood. These features of the present invention provide the benefit of significantly improving the quality of the patients' lives and thereby enhances patient compliance with the treatment regimen without reducing or modifying total daily drug dosages or eliminating treatment altogether.

This reduction in gastric distress is accomplished through the teachings of the present invention by reducing the number of daily antiviral capsules, tablets, or pills that must be taken by a patient, thereby reducing the physical and mechanical irritation to and resultant bleeding from the lining of the patient's stomach caused by the numerous ingested capsules, tablets, or pills. As a result, taking the antiviral drugs of the present invention is more tolerable to the patient undergoing antiviral therapy than the prior art drugs because nausea, vomiting, loss of appetite, and indigestion are reduced relative to known prior art antiviral therapies. Similarly, reducing the number of drug administrations required to meet the daily dosage of antiviral drug necessary to combat and resolve the viral infection at issue reduces the inconvenience and impact of the antiviral treatment regimen of the present invention on the patient's normal routine or work schedule. Thus, without modifying the antiviral drugs or the associated treatment dosing and duration protocols, the present invention enhances patient compliance by reducing the side effects associated with antiviral therapies such as ribavirin.

These advantages over the prior art are achieved through the teachings of the present invention which provides a reduced gastrointestinal side effect medicament for treating viral infection, which medicament comprises at least 500 mg of antiviral compound in an oral dosage form. Alternatively, the medicament of the present invention can comprise at least 600 mg of antiviral compound in an oral dosage form.

An exemplary antiviral compound useful for practicing the present invention is ribavirin. Ribavirin is a white, odorless white or crystalline powder. Its chemical formula is C8H12N405. It is freely soluble in water and only sparingly soluble in alcohol. It has a melting point of 166-168 degrees centigrade and is available from a number of commercial sources and generic manufacturers including Schering-Plough, Hoffman-La Roche, and Spectrum Pharmaceuticals. It ranges in price from $6.50 per 200 mg capsule to ten cents per 200 mg capsule when manufactured from bulk generic drug manufactured in China.

It again should be noted that ribavirin is not the only antiviral drug that is suitable for the practice of the present invention. Exemplary alternative antiviral compounds within the scope and teachings of the present invention include nucleoside analogues such as levovirin, a mirror image of the ribavirin molecule, viramindine, a ribavirin prodrug that is preferentially absorbed by liver cells, VX-497, an antiviral drug being developed by Vertex Pharmaceuticals, and virtually any antiviral compound causing gastrointestinal distress due to multiple oral dosing.

The oral dosage form of the medicament can be a capsule or tablet. Those skilled in the art will appreciate that “pills” come within this definition of the present invention. Where ribavirin is the exemplary antiviral drug utilized to practice the present invention dissolution of the medicament following oral administration is very fast whether the medicament is in the form of a tablet or capsule because the dissolution time in water for ribavirin powder is almost instantaneous. Thus, bioavailability of the antiviral compound is not affected by the present invention and dosages or dosing regimens need not be changed unless desired for additional reasons.

Those skilled in the art also will appreciate that by forming the medicament of the present invention with ribavirin as the antiviral compound, the medicament of the present invention is well suited for the treatment of hepatitis, regardless of virus type or subtype, and is particularly well suited for the treatment of hepatitis C, as well as for HIV infection. It also will be appreciated that as new antiviral compounds are developed, the teachings of the present invention will be equally applicable to these new compounds as well as to the existing antiviral compounds. The same is true for newly discovered viral diseases and the associated antiviral compounds developed to treat them. The teachings of the present invention are equally applicable in such circumstances.

It is also within the scope and teachings of the present invention to include one or more pharmaceutically acceptable excipients in the reduced gastrointestinal side effect medicaments. For example, microcrystalline cellulose, lactose monohydrate, croscarmellose sodium, and magnesium stearate are exemplary pharmaceutical excipients within the scope of the present invention. Other inactive carriers, excipients, lubricants or coatings are also contemplated as being within the scope and teachings of the present invention. When formed as a capsule, the medicaments of the present invention can be manufactured using gelatin capsules or other pharmaceutically acceptable capsule materials as known in the art.

Thus, an exemplary medicament illustrating the teachings of the present invention is a capsule containing 500 mg of ribavirin and intended to treat viral infections. Similarly, an alternative embodiment of the medicament of the present invention is a capsule containing 600 mg of ribavirin for use in treating viral infections. Alternatively, the 500 mg and 600 mg exemplary embodiments of the present invention can be formed as tablets. Either way, those skilled in the art will appreciate that these alternative exemplary medicaments of the present invention can be utilized to produce the same total daily dosages of antiviral compounds utilizing fewer individual capsules. For example, a 1000 mg total daily dose of ribavirin or other antiviral compound can be produced using two exemplary 500 mg capsules or tablets within the teachings of the present invention. Alternative, a 1200 mg total daily dosage of ribavirin can be produced utilizing two exemplary 600 mg medicaments of the present invention. An 1800 mg total daily dosage of ribavirin can be achieved utilizing only three of the exemplary 600 mg medicaments of the present invention.

In addition, these total daily dosages can be taken in a single administration in either the morning or the evening, or at a different time of day as is convenient to the patient's daily lifestyle and eating patterns. This simplifies the patient's life and reduces the negative and potentially discouraging psychological impact of repeated multi-dose daily dosages that can contribute to patient noncompliance. This reduction in negative psychological impact also contributes to the present inventions ability to enhance patient compliance with antiviral treatment regimens by reducing negative side effects that discourage compliance. Thus, as a result of the teachings of the present invention, the patient undergoing antiviral therapy experiences both reduced gastrointestinal side effects and reductions in the associated negative psychological side effects of the therapy. This leads to improved patient compliance relative to the prior art compounds and treatments because the patient utilizing the medicaments and teachings of the present invention is not discouraged by stomach problems every time he or she swallows a dose of antiviral compound and because the frequency and number of such potentially irritating events is also reduced.

The present invention also provides a method for enhancing a virus infected patient's compliance with a long term, multi-dose antiviral therapy regimen by reducing the gastric distress associated with antiviral therapy. This is accomplished through the steps of providing a reduced gastrointestinal side effect medicament having at least 500 mg of antiviral compound in an oral dosage form, and then administering an effective total daily dosage of the medicament to the patient for a predetermined period of time. An exemplary embodiment of this method of the present invention utilizes ribavirin as the antiviral compound with a total daily dosage ranging from 1000 mg to 2000 mg. The exemplary predetermined time ranges from six to eighteen months. This predetermined period of time is particularly useful in association with the treatment of viral hepatitis such as hepatitis C caused by HCV.

It also should be noted that both the medicaments and treatment methods of the present invention are equally well suited for use in combination antiviral therapy regimens. For example, the medicaments of the present invention can be used in combination with common or pegylated interferon, as well as any other form of interferon or combination of antiviral drugs in a combined therapy regimen for the treatment of viral diseases such as hepatitis C. Additionally, the present invention method of enhancing virus infected patient compliance with long term, multi-dose antiviral therapy can include the additional step of administering an effective weekly dosage of interferon. As those skilled in the art will appreciate, the effective weekly dosage of interferon, regardless of interferon type, can be determined within the scope and teachings of the present invention as known in the art consistent with the treatment of the viral disease at issue in the infected patient.

An additional method provided by the present invention is a method for manufacturing a reduced gastrointestinal side effect medicament for use in the treatment of a viral infection in an individual suffering therefrom. Utilizing the teachings of the present invention, such a medicament is produced in a method comprising the step of packing at least 500 mg of an antiviral compound in an oral dosage form. The dosage form can be a capsule or a tablet and the packing step can be accomplished utilizing known machines and technology to produce the appropriately sized capsule or tablet. For example, a 500 mg or a 600 mg antiviral capsule can be formed utilizing a commercially available size OO capsule and a manual or automatic capsule loading machine such as a Model PCL-V28 or PCL-H14 Automatic Capsule Loader from Pharmachem Industries and an exemplary antiviral compound such as ribavirin to fill the capsules. Similar known technology and machines can be used to form tablets or pills within the scope and teachings of the present invention.

To demonstrate the effectiveness of the present invention medicaments and methods an abbreviated clinical trial was conducted utilizing seven individuals (three male adults and four female adults) undergoing standard ribavirin/interferon combination therapy for the treatment of viral hepatitis caused by HCV. In conjunction with their respective physician, each patient was given a one week supply of the appropriate total daily dosage of reduced gastrointestinal side effect ribavirin medicament produced in accordance with the teachings of the present invention in capsule form. At the end of one week of treatment using the exemplary ribavirin medicaments and treatment methods of the present invention, including interferon administration consistent with each patient's original combination therapy protocol, each patient was given a questionnaire in connection with the confirmation of their respective pharmaceutical refills. Of the seven original patients in the study, five reported no side effects at all. One patient reported feelings of anxiety and an increased heart rate following interferon injection. Another patient reported usual side effects, but no gastrointestinal related effects. More importantly, when specifically asked, not a single patient in the study reported any gastrointestinal side effects.

Though it should be appreciated by those skilled in the art that the present invention is not limited to the treatment of hepatitis, hepatitis C, or to the treatment of RNA viruses alone, the present invention is directly applicable to the contemporary treatment of these significant viral disease conditions.

Of course the present invention is not intended to be restricted to any particular form or arrangement, or to any specific embodiment, or to any specific use, disclosed herein, since the same may be modified in various particulars or relations without departing from the spirit or scope of the claimed invention. The various exemplary embodiments hereinabove shown and described are intended only for illustration and disclosure of operative embodiments of the present invention and not to show all of the various forms or modifications in which this invention might be embodied or operated.

The present invention has been described in considerable detail in order to comply with the patent laws by providing full public disclosure of at least one of its forms. However, such detailed description is not intended in any way to limit the broad features or principles of the present invention, or the scope of the patent to be granted. Therefore, the invention is to be limited only by the scope of the appended claims.

1. A reduced gastrointestinal side effect medicament for treating a viral infection in a patient suffering therefrom comprising at least 500 mg of antiviral compound in an oral dosage form.

2. The reduced gastrointestinal side effect medicament of claim 1 wherein said antiviral compound is ribavirin.

3. The reduced gastrointestinal side effect medicament of claim 1 wherein said viral infection is hepatitis.

4. The reduced gastrointestinal side effect medicament of claim 3 wherein said hepatitis is hepatitis C.

5. The reduced gastrointestinal side effect medicament of claim 1 wherein said oral dosage form is a capsule.

6. The reduced gastrointestinal side effect medicament of claim 1 wherein said oral dosage form is a tablet.

7. The reduced gastrointestinal side effect medicament of claim 1 wherein said oral dosage form includes a pharmaceutically acceptable excipient.

8. The reduced gastrointestinal side effect medicament of claim 1 wherein said oral dosage form includes at least 600 mg or said antiviral compound.

9. The reduced gastrointestinal side effect medicament of claim 8 wherein said antiviral compound is ribavirin.

10. The reduced gastrointestinal side effect medicament of claim 9 wherein said oral dosage form is a capsule.

11. The reduced gastrointestinal side effect medicament of claim 9 wherein said oral dosage form is a tablet.

12. A method of enhancing a virus infected patient's compliance with long term, multi-dose antiviral therapy by reducing gastric distress associated therewith, said method comprising the steps of:
providing the medicament of claim 1 having at least 500 mg of antiviral compound in an oral dosage form; and
administering an effective total daily dosage of said medicament to said patient for a predetermined period of time.

13. The method of claim 12 wherein said antiviral compound is ribavirin.

14. The method of claim 13 wherein said effective total daily dosage ranges from 1000 mg to 2000 mg of ribavirin.

15. The method of claim 12 wherein said predetermined period of time ranges from six to eighteen months.

16. The method of claim 12 further including the additional step of administering an effective weekly dosage of interferon.

17. The method of claim 12 wherein said virus is HCV.

18. A method of manufacturing a reduced gastrointestinal side effect medicament for the treatment of a viral infection in an individual suffering therefrom, said method comprising the step of:
packing at least 500 mg of an antiviral compound in an oral dosage form.

19. The method of claim 18 wherein said oral dosage form is a capsule.

20. The method of claim 18 wherein said oral dosage form is a tablet.

21. The method of claim 18 wherein said antiviral compound is ribavirin.
